(19) United States
(12) Patent Application Publication
(10) Pub. No.: US 2013/0296631 A1
(43) Pub. Date: Nov. 7, 2013

(54) CLEANING ARTERIOSCLEROTIC VESSELS WITH MAGNETIC NANOSWIMMERS

(71) Applicants: Irving N. WEINBERG, (US); Lamar MAIR, (US); WEINBERG MEDICAL PHYSICS LLC, Bethesda, MD (US)

(72) Inventors: Irving N. WEINBERG, Bethesda, MD (US); Lamar MAIR, Washington, DC (US)

(73) Assignee: WEINBERG MEDICAL PHYSICS LLC, Bethesda, MD (US)

(21) Appl. No.: 13/888,684

(22) Filed: May 7, 2013

Related U.S. Application Data
(60) Provisional application No. 61/643,578, filed on May 7, 2012.

(51) Int. Cl. A61N 2/00

(52) U.S. Cl. CPC A61N 2/004; B82Y 5/00
USPC 600/12; 977/905

(57) ABSTRACT

Disclosed embodiments provide an apparatus and method for brushing plaques from vessels by exposing intraluminal nanoparticles to changing magnetic gradients.

### CROSS-REFERENCE TO RELATED APPLICATION(S)

The present application claims the benefit of priority to Provisional Patent Application No. 61/643,578 filed May 7, 2012, the contents of which are incorporated herein by reference in their entirety.

### BACKGROUND

Certain thin long nanoparticles, when immersed in a dynamic or static magnetic gradient, are known to respectively rotate and move linearly. This class of nanoparticles has been termed nanomotors or nanoswimmers. An example of the use of nanoswimmers to transport bound small molecules is given in a 2012 publication by W. Gao, D. Kagan, O. S. Pak, C. Clawson, S. Campuzano, E. Chuluun-Erdene, E. Shipton, E. E. Fullerton, L. Zhang, E. Lauga, and J. Wang, in the journal Small, volume 8(3), pages 460-467, entitled “Cargo-Towing Fuel-Free Magnetic Nanoswimmers for Targeted Drug Delivery”.

It is known that the deposition of atherosclerotic material in vessels (especially coronary and carotid arteries) is a leading cause of morbidity and mortality in developed countries. Current therapy is primarily aimed at reducing the amount of lipids circulating in the blood in order to decrease the amount of deposited materials. Efforts have been made to construct high-speed intraluminal drills that remove plaques but do not injure normal tissues, an example of which was published in 2012 by M-H Kim, H-J Kim, N. N. Kim, H-S Kim, and S-H Aim in the journal Biomedical Microdevices, volume 13, pages 963-971, entitled “A rotational ablation tool for calcified atherosclerotic plaque removal” (incorporated by reference in its entirety).

However, potential drawbacks of such tools include the possibilities of cavitation, which could affect flow to the myocardium, and the generation of large particles (e.g., greater than 10 microns) which could obstruct small vessels.

### SUMMARY

Disclosed embodiments provide an apparatus and method for brushing plaques from vessels by exposing intraluminal nanoparticles to changing magnetic gradients.

### BRIEF DESCRIPTION OF THE FIUGRES

A more complete understanding of the present invention and the utility thereof may be acquired by referring to the following description in consideration of the accompanying drawings, in which like reference numbers indicate like features, and wherein:

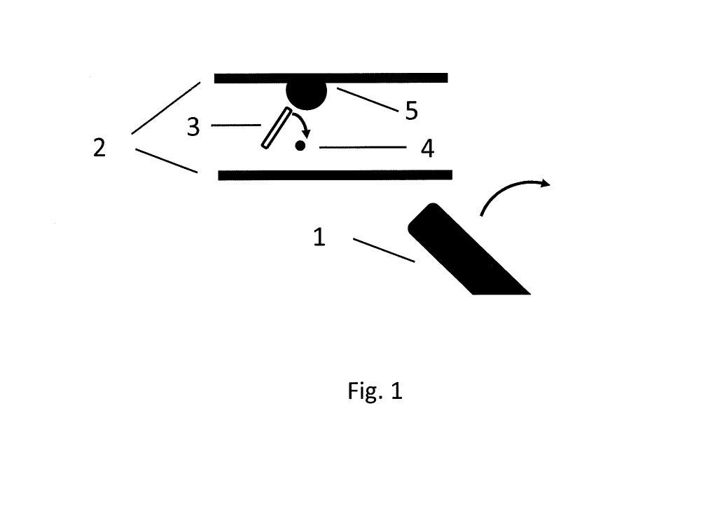

FIG. 1 illustrates apparatus including a magnetic nanoparticle and a corresponding magnet provided in accordance with the disclosed embodiments.

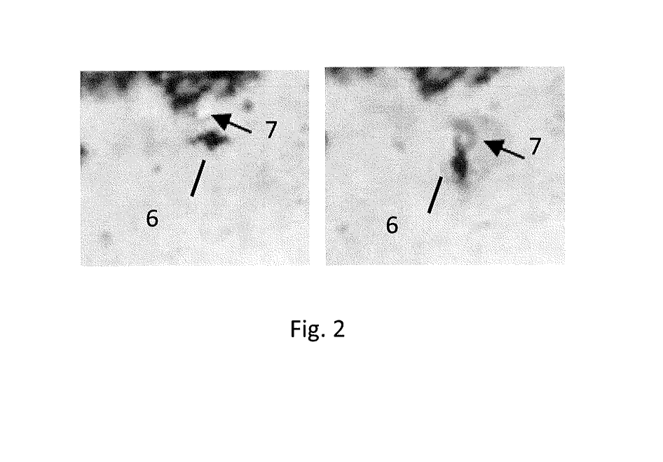

FIG. 2 includes an image of a nanoparticle assembly with a fat globule from an agglomeration of fat and water.

### DETAILED DESCRIPTION

The term “elongated” nanoparticles is used in this description to represent nanoswimmers or other nanoparticles (referred to collectively as “nanoparticles”) that rotate when immersed in magnetic gradients, or groups of such nanoparticles that may be elongated in bulk or as individual components.

In accordance with disclosed embodiments, rotatory motion of the elongated nanoparticles removes plaque material from plaque surfaces. That material can be subsequently removed through a catheter or alternatively using natural flow through the vessel.

In accordance with at least one disclosed embodiment, the nanoparticles are magnetic, have magnetic properties, and/or may be manipulated by a magnetic field. Thus, a configuration of magnetic elements (e.g., electromagnetic or permanent magnetic material) may be provided, configured and utilized to push (i.e., repel) or pull (i.e., attract) the nanoswimmers or other nanoparticles to specified locations within a patient's body, e.g., areas of vessels within the body identified via imaging (or other techniques) as suffering from plaque build-up.

It is contemplated that the nanoparticles may be steered into the desired vessel through the judicious use of magnetic gradients and forces. Thus, in accordance with at least one disclosed embodiment, an applicator is provided and inserted into a body orifice, wherein the applicator may contain a magnet to assist in transporting nanoparticles into the patient's body.

Thus, by utilizing a configuration of magnets (whether of the electromagnetic type or permanent magnets), the nanoparticles can be propelled, pushed, pulled or otherwise manipulated in relation to various anatomical and/or physiological barriers, e.g., vessel walls, to position, re-position or maintain the position(s) of the nanoparticles. This configuration of magnets may be utilized to create a local magnetic field minimum at a location distal to the barrier (as disclosed in patent publication WO2010099552, entitled “DEVICES, SYSTEMS AND METHODS FOR MAGNETIC-ASSISTED THERAPEUTIC AGENT DELIVERY,” the disclosure of which being incorporated by reference in its entirety).

The changing magnetic gradient may be produced through the rotation of a magnet near the vessel. The rotating magnet may be either of the permanent type or may be an electromagnet, or may combine components of both types. Alternatively a set of coils may be employed to implement a dynamically-changing gradient field in the vicinity of the vessel, through the use of changing electrical currents in the coils.

Disclosed embodiments may also provide a method and apparatus for manipulating the nanoparticles in a human body, for example, under imaging guidance.

For the purposes of this disclosure, the term “nanoparticles” includes particles smaller than 100 microns in any one dimension, which may be bound to chemicals or structures that have pharmacological or beneficial physical effects in the body under certain conditions, or which may have beneficial effects themselves under certain conditions (for example, to retard blood flow in an aneurysm). For the purposes of this disclosure, the term “magnetic nanoparticles” includes nanoparticles containing magnetizable materials, or which have intrinsic magnetic properties, or which may contain coils or other electrical configurations that can generate currents or voltages upon application of magnetic fields.

An apparatus designed in accordance with the disclosed embodiments may include one or more propulsive coils or sets of coils, used in conjunction with one or more electrical current generators that create pulsed magnetic gradients. These pulsed magnetic gradients may then be used to deliver the magnetic nanoparticles to desired locations in the body. The use of the term “propulsive coils” in the present application is intended to include the application of the coil for the propulsion of magnetic nanoparticles, without necessarily limiting other applications of the coils. The propulsive coils may be toroidal, planar, or of another configuration, as desired to create appropriate magnetic gradient fields for propulsion of nanoparticles in specific body locations. A planar coil configuration, for example, would be useful for manipulating magnetic nanoparticles in superficial locations of the body. An example of a planar coil configuration (used for imaging, and not for propulsion) was presented by B. Aksel, L. Marinelli, B. D. Collick, C. Von Morze, P. A. Bottomley, and C. J. Hardy, in the article entitled “Local planar gradients with order-of-magnitude strength and speed advantage,” published in 2007 by the journal Magnetic Resonance in Medicine, vol. 58, no. 1, pages 134-143 (incorporated by reference in its entirety).

The use of the term “coil” implies at least one electromagnet or electrical configuration, that may include or be used in conjunction with magnetizable materials (for example, ferrite cores) in order to produce magnetic gradient fields.

In accordance with at least one embodiment, the one or more propulsive coils may be inserted into a Magnetic Resonance Imaging (MRI) scanning system (for example, to retrofit a conventional MRI scanning system; in such an implementation the propulsive coils and other hardware and software necessary to direct the nanoparticles to the vessels of interest and/or implement the disclosed embodiment for removing plaque from blood vessel walls under direction of imaging completed by an MRI scanning system may be included in a kit for installation as part of such a retrofit or upgrade). Such a configuration was taught by the inventor in U.S. patent application Ser. No. 13/586,489 entitled “MRI-guided nanoparticle cancer therapy apparatus and methodology”, and is incorporated by reference in its entirety.

A typical MRI scanning system or scanner is a device in which the patient lies within a large, static magnet (i.e., a magnetic field that is on all of the time) where the static magnetic field is used to align the magnetization of some materials or particles in the body, and radio frequency fields are used to systematically alter the alignment of this magnetization.

In most MRI systems, the materials affected by the altered alignment are nuclear protons. In some magnetic resonance scanners, the materials affected by the altered alignment are electrons, and in other scanners the materials consist of magnetic nanoparticles. In the case where the affected materials are electrons, the MRI scanning process is often called Electron Paramagnetic Resonance Imaging (EPRI). In the case where the affected materials are electrons, the MRI scanning process is often called Magnetic Particle Imaging (MPI). The alteration in alignment causes the affected materials to produce one or more rotating magnetic fields that are detectable by the scanner. The detected rotation of the magnetic fields is recorded to construct an image of the scanned area of the body. Magnetic field gradients cause affected materials at different locations in space to rotate at different speeds. By applying magnetic field gradients in different directions, 2D images or 3D volumetric images can be obtained in many orientations.

MRI can provide good contrast between the different soft tissues of the body, which makes it especially useful in imaging the brain, muscles, the heart, and cancers. Unlike CT scans or traditional X-rays, MRI does not use ionizing radiation.

It should be understood that the propulsive coil(s) provide the ability to push nanoparticles from their initial positions to various locations in the body, as well as to pull the nanoparticles from various initial locations to other locations. Moreover, these propulsive coil(s) also provide the ability to both agitate, e.g., rotate, the nanoparticles as well as stabilize the nanoparticles in their locations.

Prior work in manipulation of nanoparticles with magnetic gradients employed permanent magnets held near the body part, as taught in the article by A. S. Lübbe entitled “Clinical Experiences with Magnetic Drug Targeting: A Phase I Study with 4′-Epidoxorubicin in 14 Patients with Advanced Solid Tumors,” published in the journal Cancer Research, volume 56, pages 4686-4693, on Oct. 15, 1996 (and incorporated by reference in its entirety). The use of permanent magnets in such a manner would not be possible in a typical MRI system, due to the large forces that would be applied on the permanent magnets by the MRI static field, and the interference by the permanent magnets in the magnetic gradient pulses that are used by the MRI scanner to form an image. The magnetic gradient pulses that are used by the MRI scanner typically have a maximum magnitude of 40 mT that is applied over a distance of 70 cm, which is not strong enough to move the Nanoparticles. Applying pulsed magnetic gradients with higher magnitudes has been conventionally difficult because of the resulting nerve stimulation caused by induced magnetic fields, as discussed by P. Mansfield and P. R. Harvey in an article entitled “Limits to neural stimulation in echo-planar imaging,” published in the journal Magnetic Resonance in Medicine, vol. 29, number 6, pages 746-758, in 1993 (incorporated by reference in its entirety).

From the above considerations, one can determine that it would be difficult if not impossible to manipulate and agitate nanoparticles within an MRI system using conventionally known methods. This difficulty is problematic because a physician may prefer to visualize the concentration of nanoparticles within the body in the course of their manipulation. Thus, the present invention addresses the challenge of manipulating nanoparticles under MRI guidance by utilizing pulsed magnetic gradients to propel or agitate the nanoparticles.

In accordance with at least one embodiment, the pulsed magnetic gradients created by the propulsive coil(s) are not contemporaneous with the pulsed magnetic gradients used by the MRI system to create an image of the body and/or nanoparticles in the body. For example, the propulsive magnetic gradient pulses are interleaved with the magnetic gradients used for imaging purposes, or may precede or follow the magnetic gradients used for imaging purposes. This lack of contemporaneity implies that the pulsed magnetic fields used to propel the nanoparticles do not interfere with the process of collecting an image with the MRI scanner, where “interference” is defined for the purposes of this description as a process that would cause reduced quality of the MRI scanner image.

In at least one alternative embodiment, strong pulsed magnetic gradients are used to propel nanoparticles and also as part of the process of creating an image of the body and/or nanoparticles in a patient's body. Unlike the prior art, in which the magnetic gradients used to create an image are of low magnitude, at least one presently disclosed embodiment employs features disclosed in U.S. Pat. No. 8,154,286, by the present inventor, Irving Weinberg, entitled “Apparatus and method for decreasing bio-effects of magnetic fields”, issued Apr. 10, 2012 (and incorporated by reference in its entirety), and published in the scientific literature in an article by I. N. Weinberg, P. Y. Stepanov, S. T. Fricke, R. Probst, M. Urdaneta, D. Warnow, H. Sanders, S. C. Glidden, A. McMillan, P. M. Starewicz, and J. P. Reilly, entitled “Increasing the oscillation frequency of strong magnetic fields above 101 kHz significantly raises peripheral nerve excitation thresholds,” in a May 2012 article in the journal Medical Physics, vol. 39, no. 5, pages 2578-83 (and incorporated by reference in its entirety). By employing one or more magnetic gradient pulses with very short rise-times and/or fall-times (for example, less than 100 microseconds) as disclosed in U.S. Pat. No. 8,154,286, the magnitude of the magnetic gradients can be at least ten times higher than in the prior art (for example, 400 milliTeslas). Such high magnitudes would be similar to those previously obtained with permanent magnets for manipulating nanoparticles, as in the above-cited publication by Lübbe et al. Thus, the same coils used to produce propulsion can be used to create an image in the MRI scanner. As discussed above, the process of creating an image in an MRI scanner includes the alteration of rotational frequencies of materials in the body, through the application of pulsed magnetic gradients, typically by modifying the resonant frequencies of polarizable particles in a space-dependent manner. The use of propulsive coils to both propel MNPs and collect images with the MNI scanner implies that the pulsed magnetic fields used to propel the MNPs do not interfere with the process of collecting an image with the MRI scanner.

In accordance with at least one presently disclosed embodiment, a pulsed magnetic gradient may be applied by the propulsive coil(s) to materials in a patient's body in order to increase magnetization of the materials, prior to the application of other sequences of pulsed magnetic gradients. This use of a prior pulse is termed “pre-polarization”, and is taught by U.S. patent application Ser. No. 12/888,580, having Irving Weinberg as an inventor and entitled, “ULTRA-FAST PRE-POLARIZING MAGNETIC RESONANCE IMAGING METHOD AND SYSTEM” (incorporated by reference in its entirety). The use of propulsive coils to both propel MNPs and increase magnetization of materials within the MRI scanner implies that the pulsed magnetic fields used to propel the MNPs do not interfere with the process of collecting an image with the MRI scanner.

Thus, various mechanisms may be provided for accurately and effectively agitating, moving or rotating the nanoparticles to effect plaque removal from vessel walls. The term “rotation” is used to refer to a motion that will enable or accelerate removal of plaque, for example to-and-fro swiveling or full angular rotation around the short or long axis of the nanoparticle, or other such motions.

An embodiment of the apparatus is shown in FIG. 1, where magnet 1 rotates near a vessel having walls 2. A long nanoparticle 3 is thereby caused to rotate, removing a plaque portion 4 from an atherosclerotic plaque 5 located on the vessel wall 2.

FIG. 2 shows an image of a nanoparticle assembly that has grabbed a fat globule from an agglomeration of fat and water.

It is contemplated that the use of the long nanoparticles is combined with direct visualization of the vessels, plaques, and or nanoparticles through magnetic resonance imaging. The application of changing gradients may be implemented through the use of the native MRI system's gradient coils, or may be implemented though add-on coils capable of creating strong gradient fields as in prior U.S. Pat. No. 8,154,286 (“Apparatus and method for decreasing bio-effects of magnetic fields”) by one of the inventors of the present patent. As described in that prior invention, it is possible to create strong gradient fields without untoward nerve stimulation through the use of rapid rise- and fall-times. Alternatively, the magnetic gradients for rotating the nanoparticles can be created through a planar MRI system, whose magnetic fields are strong enough to penetrate into the heart.

It is contemplated that the nanoparticles may be coated with chemicals to aid in the removal of plaque. Examples of such chemicals are bile salts and cholesterol ester oils, as disclosed in the 1978 publication by B. E. North, S. K. Katz, and D. M. Small in the journal Atherosclerosis, volume 30, pages 211-217, entitled “The Dissolution of Cholesterol Monohydrate Crystals in Atherosclerotic Plaque Lipids”.

While this invention has been described in conjunction with the specific embodiments outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

What is claimed:

1. A system for reducing plaque in a vessel, the system comprising:
at least one nanoparticle introduced into a vessel;
at least one magnet positioned in proximate relationship to the vessel and manipulated to induce motion of the at least one nanoparticles in the vessel as a result of exposure to changing magnetic gradients resulting from manipulation of the at least one magnet,
wherein the induced motion of the at least one nanoparticle causes a reduction in volume of plaque located in and/or located on walls the vessel.

2. The system of claim 1, wherein manipulation of the at least one magnet comprises changing direction and/or magnitude of at least one magnetic field produced by the at least one magnet.

3. The system of claim 1, wherein the reduction in volume results in removal of unwanted substances from a vascular wall or lumen.

4. The system of claim 1, wherein the at least one nanoparticle is coated with a chemical that aids in the reduction of plaque located in and/or on the walls of the vessel.

5. The system of claim 1, wherein the at least one nanoparticle includes nanoswimmers.

6. The system of claim 1, wherein the at least one nanoparticle includes Janus particles.

7. The system of claim 1, wherein the vessel is a blood vessel in a human body.

8. A device comprising:
a plurality of small magnetizable particles,
wherein introduction of the plurality of particles into one or more vessels along with subsequent manipulation of a magnetic field applied to the one or more vessels results in motion of the plurality of particles that causes a reduction in volume of plaque located in and/or located on walls of the one or more vessels.

9. The device of claim 8, wherein manipulation of the magnetic field comprises changing direction and/or magnitude of the magnetic field.

10. The device of claim 8, wherein the reduction in volume results in removal of unwanted substances from a vascular wall or lumen.

11. The device of claim 8, wherein the at least one nanoparticle is coated with a chemical that aids in the reduction of plaque located in and/or on the walls of the vessel.

12. The device of claim 8, wherein the at least one nanoparticle includes nanoswimmers.

13. The device of claim 8, wherein the at least one nanoparticle includes Janus particles.

14. The device of claim 8, wherein the one or more vessels are blood vessels in a human body.

15. A method of reducing plaque in a vessel, the method comprising:
introducing at least one nanoparticle into a vessel; and
manipulating a magnetic field produced by at least one magnet positioned in proximate relationship to the vessel to induce motion of the at least one nanoparticle in the vessel as a result of exposure to changing magnetic gradients resulting from the manipulation of the at least one magnet,
wherein the induced motion of the at least one nanoparticle causes a reduction in volume of plaque located in and/or located on walls the vessel.

16. The method of claim 15, wherein manipulation of the magnetic field comprises changing direction, magnitude, or frequency of the magnetic field.

17. The method of claim 15, wherein the reduction in volume results in removal of unwanted substances from a vascular wall or lumen.

18. The method of claim 15, further comprising, coating the at least one nanoparticle with a chemical that aids in the reduction of plaque located in and/or on the walls of the vessel, prior to introduction of the at least one nanoparticle into the vessel.

19. The method of claim 15, wherein the at least one nanoparticle includes nanoswimmers.

20. The method of claim 15, wherein the at least one nanoparticle includes Janus particles.

21. The method of claim 15, wherein the vessel is a blood vessel in a human body.

22. The method of claim 15, wherein manipulation of the magnetic field so as to induce motion of the at least one nanoparticle is conducted under imaging guidance.

23. The method of claim 15, further comprising heating the at least one nanoparticle using radiofrequency absorption.

24. The method of claim 15, further comprising heating at least one nanoparticle by application of alternating magnetic fields.
